(19) United States
(12) Patent Application Publication
(10) Pub. No.: US 2011/0301486 A1
(43) Pub. Date: Dec. 8, 2011

(54) MEASUREMENT OF AUDITORY EVOKED RESPONSES

(75) Inventors: Dirk Van Hek, Eindhoven (NL); Christoph Van Der Reijden, Best (NL)

(73) Assignee: CORDIAL MEDICAL EUROPE, Eindhoven (NL)

(21) Appl. No.: 12/793,064

(22) Filed: Jun. 3, 2010

(51) Int. Cl. A61B 5/0484

(52) U.S. Cl. 600/544

(57) ABSTRACT

The invention relates to a method of measuring an electroencephalogram signal in response to an auditory stimulus applied to a subject or patient (99), wherein the method uses at the most a first electrode (EL1), a second electrode (EL2), a third electrode (EL3). The method comprises: i) positioning the first electrode (EL1) on the head (10) of the patient or subject (99) in a first region (R1) extending substantially between a right ear and a right eye; ii) positioning the second electrode (EL2) on the head (10) of the subject or patient (99) in a second region (R2) extending substantially between a left ear and a left eye; iii) positioning the third electrode (EL3) on the head (10) of the subject or patient (99) in a third region (Cz), the third region (Cz) being different from the first region (R1) and the second region (R2); iv) applying the auditory stimulus to at least one ear of the subject or patient (99); v) measuring a first potential difference between the first electrode (EL1) and the third electrode (EL3) in response to the auditory stimulus to obtain a first electroencephalographic signal, and measuring a second potential difference between the second electrode (EL2) and the third electrode (EL3) in response to the auditory stimulus to obtain a second electroencephalographic The invention further relates to a method of hearing screening a subject or patient, comprising such method. The invention also relates to a measurement system (such as a hearing screener) for carrying out such method and to an apparatus (head-set) for use in such system. The invention provides for an improved method of measuring an electroencephalogram signal in response to an auditory stimulus applied to a subject or patient, in particular where two channels are measured using only three electrodes.

### FIELD OF THE INVENTION

The invention relates to a method of measuring an electroencephalogram signal in response to an auditory stimulus applied to a subject or patient, wherein the method uses at the most a first electrode, a second electrode, a third electrode. The invention also relates to a hearing screen method comprising such method. The invention further relates to a measurement system for carrying out such method, and to a hearing screener comprising such measurement system. The invention also relates to an apparatus for use in such measurement system or hearing screener.

### BACKGROUND OF THE INVENTION

Methods of measuring auditory evoked responses on the head of a patient are known, for instance from the technical field of hearing screening. Hearing is an essential feature to enable communication and to enjoy music. The ability to hear is also essential to develop language and to understand speech. Although it is not until the age of four years that a child is able to speak in grammatically correct sentences, auditory perception skills undergo significant development before the age of six months (Yoshinaga-Itano et al., “Language of early—and later—identified children with hearing loss”, Pediatrics, 1998, v102, p1161-1171). This has resulted in the introduction of universal neonatal hearing screening programs in a number of countries. Two types of hearing screeners are used in these neonatal hearing screening programs.

A first type of hearing screener uses a probe that is put into the outer ear canal. This probe contains a loudspeaker and a microphone. The microphone listens to emissions of sound from the cochlea in response to a sound that is delivered to that ear by the loudspeaker. This is called oto-acoustic emissions (OAE). This technique is cheap, but has a limited sensitivity in detecting children with a possible hearing loss: A pass means that the cochlea is OK, but the integrity of the neural pathway after the cochlea leading to the auditory cortex is not tested. This is a big disadvantage of the OAE.

The second type of hearing screener tests the whole pathway (i.e. the acoustic and the neurological pathway): A sound stimulus (usually clicks) is delivered to the outer ear canal by a transducer (headphone, insert ear phone) and electrodes on the human head record the responses from the brainstem that are in synchrony with the stimulus. This method is called the Automated Auditory Brainstem Response (AABR). This method is the gold standard of hearing screening (J. W. Hall, New Handbook of Auditory Evoked Responses. 2007).

U.S. Pat. No. 5,230,344, the contents of which is incorporated by reference in its entirety, discloses an evoked potential processing system which includes, in one embodiment, a spectral averaging method. Time based, digital pre-stimulus and post-stimulus electroencephalographic (EEG) signal streams are obtained and are converted into frequency spectrum signals. A differential spectrum is obtained. The differential spectrums from a plurality of sweeps are summed. The summed differential spectrum is then converted into a time based signal stream which contains the evoked potential (EP) signal therein. The EP signal can also be obtained utilizing a two-dimensional filter. Pre- and post-stimulus EEG signal streams for a sub-group of stimuli, wherein each stimulus in a group has the same intensity or frequency, are filtered by conventional averaging or spectral differential averaging. The time based, filtered, post-stimulus EEG signal streams are placed in an array and the array is then filtered by a two-dimensional Fast Fourier Transform (FFT) filter. The array is then filtered by a mask and the masked array is then transformed into a time based format by an inverse FFT. The adaptive averaging technique utilizes a computational formula which computes an estimated running signal to noise ratio. When the difference between the pre-stimulus running SNR and the post-stimulus running SNR is less than a predetermined threshold, further stimulation and acquisition of EEG signals stops. Hence, the post-acquisition processing of the EEG signals is limited to that number of EEG sweeps. The electrode wire configuration uses a cross wiring scheme wherein the shield of a particular wire is connected to the other electrode wire to eliminate artifacts in the respective electrode wire.

While U.S. Pat. No. 5,230,344 shows an electrode wire configuration to eliminate or reduce noise or artifacts in the electrode pick up wiring. U.S. Pat. No. 5,099,856, the contents of which is incorporated by reference in its entirety, shows to additionally connect at least one of the electrodes to the power supply to reduce common mode noise and artifacts. However, it has been found by the inventors of the present system, that these configurations surprisingly reduce common mode rejection and thereby, increases noise and artifacts.

The next paragraphs describe different hearing screeners, based on the AABR technique. Most of them have the additional functionality of diagnosing hearing thresholds i.e. determine hearing thresholds (in dB) as a function of sound frequency (e.g. 0.5, 1, 2 and 4 kHz) also called an audiogram.

Intelligent Hearing Systems (IHS, Miami, Fla., USA, information available at www.intelligenthearingsystems.com) sells the “SmartScreener”. This product is a hearing screener with the option of hearing diagnostics. This product uses a personal computer or laptop to run software for hearing screening or hearing diagnostics. A universal smart box producing the auditory stimuli is connected to this personal computer or laptop. A second box containing at least two amplifiers is also connected to this personal computer or laptop. In a one-channel EEG recording configuration, at least three electrodes are connected to the amplifier-box by electrical wires. In a two-channel EEG recording configuration, at least four electrodes are connected to the amplifier-box (see their smart notes on their website about the ABR Screening using the Smart Screener). Connecting three electrodes to the skin in a one-channel EEG recording configuration increases preparation time. Connecting four electrodes in a dual-channel configuration to the skin increases even more the preparation time. Furthermore an increased number of electrodes increases the risk of bad electrode contact to the skin of the subject during hearing screening which increases testing time.

Interacoustics (Assens, Denmark, information available at www.interacoustics.dk) sells the ABRIS. This product is a hearing screener with the option of hearing diagnostics. The software of this product is based on scientific research of the German researchers: E. Stürzebecher and M. Cebulla. This product uses a personal computer or laptop to run software for hearing screening or hearing diagnostics. A box called the “Eclipse Platform” utilized to produce the auditory stimuli is connected to this personal computer or laptop. A second box containing at least two amplifiers is also connected to this personal computer or laptop. In a one-channel EEG recording configuration, at least three electrodes are connected to the amplifier-box by electrical wires. In a two-channel EEG recording configuration, at least four electrodes are connected to the amplifier-box (the Eclipse Operating Manual is available at http://www.interacoustics.com/com_en/Pages/Product/Abr/EvokedPotentials.htm?pr odid=60808). Connecting three electrodes to the skin in a one-channel EEG recording configuration increases preparation time. Connecting four electrodes in a dual-channel configuration to the skin increases even more the preparation time. Furthermore an increased number of electrodes increases the risk of bad electrode contact to the skin of the subject during hearing screening which increases testing time.

Maico-Diagnostic GmbH (Berlin, Germany, information available at www.maico-diagnostic.com) sells the Beraphone. This product is a hearing screener with the option of hearing diagnostics. The software is based on scientific research of the German researchers: E. Stürzebecher and M. Cebulla. The product comprises a unit, the Beraphone, which is placed on one of the ears. It produces sounds and a one-channel EEG is amplified. In this unit, three reusable electrodes are integrated to pick up the electrophysiological signals. This unit is connected to a personal computer or laptop on which software runs to evaluate the amplified one-channel EEG for responses to the auditory stimuli.

WO03/032811A2, the contents of which is incorporated by reference in its entirety, shows an apparatus and method for evaluation of hearing loss. The apparatus and method use evoked auditory brainstem responses (ABR) to determine if the subject is able to hear repeatedly administered click stimuli. In order to optimize evaluation, the apparatus uses normative data that is age dependent to weight the auditory responses, and to compensate for different or changing noise conditions. However, as such, the apparatus is complex to use.

As of 2006 hearing screening has been implemented in the USA, Belgium(Flanders), The Netherlands and in other countries. Two techniques have been developed to screen the ears, one is testing the Cochlea only by recording oto-acoustic emissions (OAEs), and the other technique tests the cochlea plus the neural pathway till the brainstem and is called Auditory Brainstem Response (ABR). An automated version for hearing screening is called Automated Auditory Brainstem Response (AABR). Basically, short audible clicks of about 1 msec in length are delivered to the outer ear canal. Responses to these clicks are calculated from a one-channel Electro-Encephalogram (EEG) by averaging and comparing the shape of this average to a reference or template. The average response is a characteristic ABR-waveform within a time window of about 0-12 msec after the onset of the stimulus. Common electrode sites to record these responses are electrode positions on the midline of the head: Forehead- neck or Vertex-neck (New Handbook of Auditory Evoked Respones. J. W. Hall III, 2007).

Another way of hearing screening is based on recording the Auditory Steady-State Response. This technique uses a similar hardware setup, as the ABR, but differs among auditory stimuli applied to the ears and differs among the type of analysis of the recorded Electro-Encephalo-Gram (EEG)-signals. Recent developments in hearing screening have led to inventions which are claimed in (non-prepublished) U.S. patent application Ser. No. 12/412,343, with the title: “Hearing Screening System for a Subject or a Patient, and a Method for Hearing Screening”, filed on 26 Mar. 2009. A corresponding PCT application PCT/EP2010/053923 was filed on 25 Mar. 2010. Both patent applications are hereby incorporated by reference in their entirety.

These patent applications disclose a hearing screener basically comprising a headphone combined with a 2-channel EEG (Electro-Encephalo-Gram) recorder. This device has been described in detail in our U.S.-patent application with Ser. No. 12/412,343. In short, this hearing screener operates as follows. The loudspeakers in the headphone produce 90 Hz amplitude modulated sounds simultaneously, which are delivered to the outer ear-canal of the subject. In normal hearing subjects, these sounds are transformed by the cochlea to electrical signals and delivered to the hearing nerves. In the brainstem and in the auditory cortex, neural generators produce the response EEG-signal to the sounds that are delivered to the ears. These EEG-signals are recorded with a 2-channel EEG recorder from the following electrode positions: Left Mastoid—Vertex and Right Mastoid—Vertex. The EEG is averaged and a Fast Fourier Transform is applied to this average. A statistical test (F-test) compares the EEG response amplitude at 90 Hz with the EEG noise in the neighboring frequencies.

### SUMMARY OF THE INVENTION

It is a first object of the invention to provide further improvements in measuring an electroencephalogram signal in response to an auditory stimulus applied to a subject or patient, in particular when being measured with only two channels using only three electrodes. It is a second object of the invention to provide a hearing screening method in which such method is used. It is a third object of the invention to provide a measurement system for carrying out such method. It is a fourth object of the invention to provide a hearing screener comprising such measurement system. It is a fifth object of the invention to provide an apparatus (such as a head-set) for use in the measurement system of the invention.

The invention is defined by the independent claims. The dependent claims define advantageous embodiments.

In a first aspect, in accordance with the first object, the invention relates to a method of measuring an electroencephalogram signal in response to an auditory stimulus applied to a subject or patient, wherein the method uses at the most a first electrode, a second electrode, a third electrode. The method comprises:

positioning the first electrode on the head of the patient or subject in a first region extending substantially between a right ear and a right eye;

positioning the second electrode on the head of the subject or patient in a second region extending substantially between a left ear and a left eye;

positioning the third electrode on the head of the subject or patient in a third region, the third region being different from the first region and the second region;

applying the auditory stimulus to at least one ear of the subject or patient;

measuring a first potential difference between the first electrode and the third electrode in response to the auditory stimulus to obtain a first electroencephalographic signal, and

measuring a second potential difference between the second electrode and the third electrode in response to the auditory stimulus to obtain a second electroencephalographic signal.

The effect of the features of the invention is as follows. In (non-prepublished) U.S. patent application Ser. No. 12/412,343 a 2-channel, 3-electrode, measurement technique is described. In an embodiment therein it is prescribed that the first and second electrodes are positioned on the mastoid positions behind the ears, whereas the third electrode is advantageously placed on the Cz position on the head of the subject or patient. Such three-electrode configuration may be advantageously integrated in a head-set extending to both ears and crossing the Cz position on the head. The inventor has discovered that such electrode positioning may be disadvantageous in certain cases, in particular when applied to babies. Experiments have shown that the placement of the electrodes, such as disposable electrodes or durable electrodes (made of titanium for example), on the Mastoid locations can be cumbersome. For good measurement of the electroencephalogram signal it is required that the electrodes make good electrical contact with the Mastoid positions, but this was not always the case. It appeared that it is sometimes very difficult to get the electrodes on the right spot, i.e. on the Mastoids of the baby. This was mainly because the Mastoids of a baby have limited size, and secondly because the part of the scalp parietal to the Mastoids, has a curved shape instead of a flat surface needed to make secure electrical contact between electrodes and the scalp. In other words, the electrode-surface did not always produce a good electrical contact to the scalp of the baby and therefore the signals were not always recorded well. The inventor has realized that this problem can be prevented by placing the electrodes in a region before the ears instead of behind the ears (Mastoid positions). By doing so there is still the benefit of easy integration with a headset extending over both ears. The improved placement of the electrodes is because the part of the scalp between the ears and the eyes has a more or less flat shape. This makes it easy to put the electrodes there and make them stick there for at least 10-15 minutes. However, on top of that, it has become much easier to establish a good electrical contact between the electrode and the head of the subject or patient which has a positive impact on the integrity of the measured signals obtaining a good quality 2-channel electroencephalogram.

Another advantageous effect of the invention is that the electrodes can be monitored continuously by visual inspection during the whole recording session when the subject or patient is lying on his or her back.

It must be noted that the invention is intended for, but not necessarily restricted to, healthy persons having two ears and two eyes without substantive anatomic deviations.

In an embodiment of the method in accordance with the invention at least one of the first region and the second region is further defined as a fictitious strip having a midline, the midline extending from an external auditory meatus (also being referred to as outer ear canal, auditory duct, and auditory canal) of the respective ear in a direction towards a nose. Throughout the description the word “strip” must be interpreted as an elongate shape, but not necessarily having a 100% rectangular shape. Here it must be noted that the strip is defined with respect to a projection of the head on a flat surface. The height of the ear does not necessarily need to be equal to the vertical height of the nose. This embodiment further restricts the size of the regions to a strip-shaped region having a height, which is substantially constant over its length (it must be kept in mind that the face of a patient is not flat, but follows a 3-dimensional contour, differing from subject to subject). The strip is defined as a part of this surface. The midline which runs from the external auditory meatus (in projection on the flat surface) to the nose may extend in a substantial horizontal direction, but this is not necessary and also depends on the anatomy of the subject or patient. In any case this embodiment defines a region on the head where the placement of the electrodes is easier and provides a better contact, in particular because the surface is relatively flat within the strip.

In an embodiment of the method in accordance with the invention the strip has a width of 6 centimeters, and preferably 4 centimeters, and even more preferably 2 centimeters. Placement of the electrodes is easier and more effective within the region in accordance with this embodiment.

In an embodiment of the method in accordance with the invention a heart-heart distance between each respective one of the first and second electrodes and the respective external auditory meatus lies in the range between 1 and 2 centimeters. Experiments have shown that this region is the best for placement of the electrodes.

In an embodiment of the method in accordance with the invention the third electrode is positioned on the midline of the head of the subject or patient. Placing the third electrode on the midline of the head has the advantage that the integration with the first and the second electrode in a frame or head-set (that can be put on the head to fix locations of the electrodes) becomes easier. The midline follows the contour of the head and extends from the forehead to the neck of the subject or patient.

In an embodiment of the method in accordance with the invention the third electrode is positioned on a Cz position of the head. The Cz position (which definition is well-known in the field) is very advantageous because it substantially coincides with a location right above the ears, further facilitating integration of the electrodes in a frame or head-set that can be put on the head. A further advantage of using the Cz position is that the signal-to-noise ratio of the evoked potentials is optimized (see also the earlier reference to “van der Reyden”).

An embodiment of the method further comprises amplifying a first potential difference between the first electrode and the third electrode. An embodiment of the method further comprises amplifying a second potential difference between the second electrode and the third electrode. Amplification of the signals facilitates further processing of signal, for instance for hearing screening purposes.

In a second aspect, in accordance with the second object, the invention relates to a method of hearing screening a subject or patient, the hearing screening method comprising the method of the invention. The invention is particularly advantageous in the field of hearing screening, wherein a limited number of EEG channels is recorded and processed. Each recorded channel should provide a decent signal or else the method of hearing screening may fail.

In a third aspect, in accordance with the third object, the invention relates to a measurement system for carrying out the method of the invention.

An embodiment of the measurement system in accordance with the invention further comprises a frame, and the respective electrodes, the respective electrodes being mounted to the frame, wherein the frame is further configured for fixing respective positions of the respective electrodes in the respective regions in operational use.

An embodiment of the measurement system in accordance with the invention further comprises an auditory stimulus device for applying the auditory stimulus to the at least one ear of the subject or patient, the auditory stimulus device being mounted to the frame. The addition of an auditory stimulus device to the measurement system facilitates hearing screening measurements. The stimulus device is configured for producing a sound, which, when heard by the subject or patient, results in a detectable response in the EEG signals.

In a fourth aspect, in accordance with the fourth object, the invention relates to a hearing screener comprising the measurement system according to the invention. The hearing screener further comprises an amplifier for amplifying the electroencephalogram signals, and a processor unit connected to the auditory stimulus device for controlling the application of the auditory stimulus, the processor unit being further configured for receiving, collecting, and processing data from the amplifier to obtain quantitative data about the hearing ability of the subject or patient.

In a fifth aspect, in accordance with the fifth object, the invention relates to an apparatus for use in the measurement system of the invention. The apparatus further comprises the frame, the first electrode, the second electrode, and the third electrode. The apparatus may be called a head-set. In any case the frame ensures that the relative positions of the electrode are fixed and when put on the head of the subject or patient it further ensures that the electrodes are kept at the right position on the head.

An embodiment of the apparatus in accordance with the invention comprises an auditory stimulus device for applying the auditory stimulus to the at least one ear of the subject or patient. The auditory stimulus device may be advantageously integrated in the same frame as the electrodes.

In an embodiment of the apparatus in accordance with the invention the auditory stimulus device has been mounted to the frame.

In an embodiment of the apparatus in accordance with the invention at least one of the respective electrodes comprises an electrically conductive shield being electrically insulated from the at least one of the respective electrodes and covering a substantial part of the at least one of the respective electrodes and extending substantially to a contact surface of the at least one of the respective electrodes, wherein the electrically conductive shield is connected to a reference potential of the measurement system. Experiments with a hearing screener of as described in U.S. application Ser. No. 12/412343 have revealed that mains power supplies at a distance of about 2.5 meters and less to our hearing screener, contributed significantly to 50 Hz noise. In fact these 50 Hz noise amplitudes were so large that they made hearing screening very difficult, if not impossible. The inventor was able to reproduce this electromagnetic noise interference by recording electrophysiological signals in the presence and absence of a mains cable that was plugged into the mains power supply. The distance of this mains cable to the hearing screener was less than 50 cm. Further experiments revealed that the common mode suppression is particularly not very effective if the distance between the hearing screener and the EM-noise source is less than about 10 times the distance between the electrodes (=about 10 cm). The embodiment described here overcomes this problem. The shield forms a Faraday cage around the electrodes. The shield may be designed such that it covers almost the full electrode develops into the shielding of the wiring which connects the electrode to further circuitry. More detailed information is given in the detailed description of the embodiments. Preliminary recordings on newborn babies have confirmed that the mains power noise is effectively removed from the EEG signals in this embodiment. As a consequence of this embodiment the noise on the measured signals is reduced, which results in a higher reliability of the measurement, and optionally a higher success rate of the hearing screening method (and system).

These and other aspects of the invention are apparent from and will be elucidated with reference to the embodiments described hereinafter.

### BRIEF DESCRIPTION OF THE DRAWINGS

In the drawings:

FIG. 1 shows parts of a hearing screening system in accordance with an embodiment of the invention;

FIGS. 2a to 2d show different views of a head-set as part of a hearing screening system in accordance with another embodiment of the invention;

FIGS. 3a to 3d show different views of a remote-control to be coupled to the head-set of the hearing screening system of FIGS. 2a to 2d;

FIGS. 4a to 4c shows regions on a head of a subject of patient on which EEG measurement electrode are placed in accordance with different embodiments of the invention, and

FIG. 5 shows a shielded electrode in accordance with yet another embodiment of the invention.

### LIST OF REFERENCE NUMERALS

10 head of patient or subject
50 adjustment device for third electrode
HB flexible head-band
SPL speaker left
SPR speaker right
EC ear-caps
H hinges
EL1 first electrode
EL2 second electrode
EL3 third electrode
PS1 first power supply (galvanically isolated from second and third power supplies)
PS2 second power supply (galvanically isolated from first and third power supplies)
PS3 third power supply (galvanically isolated from first and second power supplies)
DA1 first differential amplifier (part of pre-amplifier stage, channel 1)
DA2 second differential amplifier (part of pre-amplifier stage, channel 2)
GND1 intermediate supply voltage of first power supply (i.e. ground or 0V)
VDD1 first supply voltage (potential) of first power supply (i.e. +15V)
VSS1 second supply voltage (potential) of first power supply (i.e. −15V)
GND2 intermediate supply voltage of second power supply (i.e. ground or 0V)
VDD2 first supply voltage (potential) of second power supply (i.e. +15V)
VSS2 second supply voltage (potential) of second power supply (i.e. −15V)
GND3 intermediate supply voltage of third power supply (i.e. ground or 0V)
VDD3 first supply voltage (potential) of third power supply (i.e. +15V)
VSS3 second supply voltage (potential) of third power supply (i.e. −15V)
C11 first coupling capacitor on first input of first differential amplifier
C12 second coupling capacitor on second input of first differential amplifier
C21 first coupling capacitor on first input of second differential amplifier
C22 second coupling capacitor on second input of second differential amplifier
IA1 first isolation amplifier (channel 1)
IA2 second isolation amplifier (channel 2)
IS input side of isolation amplifiers
OS output side of isolation amplifiers
GB galvanic barrier of isolation amplifiers
CH1 first channel
CH2 second channel
HS head-set (flexible head-band)
FR frame
CD control device/remote control
TSC touch screen with displayed control buttons
LG logo
CBL cable coupled to head-set
99 subject or patient
R1 first region for first electrode (strip-shaped)
R2 second region for second electrode (strip-shaped)
ML midline of strip
R1′ first (further restricted) region in accordance with a further Embodiment (strip-shaped)
R2′ second (further restricted) region in accordance with a further embodiment (strip-shaped)
R1″ first (even further restricted) region in accordance with a further advantageous embodiment
R2″ second (even further restricted) region in accordance with a further advantageous embodiment
SAE sensing area of electrode
SH electrically conductive shield (electromagnetic shield)
DL electric isolation/dielectric
WR connecting wire (coupling electrode to amplifier)

### DETAILED DESCRIPTION OF THE PREFERRED EMBODIMENT

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well known devices, circuits, tools, techniques and methods are omitted so as not to obscure the description of the present system. It should be expressly understood that the drawings are included for illustrative purposes and do not represent the scope of the present system.

The invention relates to an improved method of measuring an electroencephalogram signal in response to an auditory stimulus applied to a subject or patient. Such measuring was part of a hearing screening method as disclosed in U.S.-patent application Ser. No. 12/412343. The method described therein requires only three electrodes (but more may be used optionally). The hearing screener comprises a headphone (head-set) combined with a 2-channel EEG (Electro-Encephalo-Gram) recorder. In short, it this apparatus operates as follows.

In an embodiment the loudspeakers in the headphone produce 90 Hz amplitude modulated sounds simultaneously (but this may also be another frequency, for example between 80 and 100 Hz), which are delivered to the outer ear-canals of the subject and the frequencies applied to each ear may differ a few Hz. More information about this is given later in the description. In normal hearing subjects, these sounds are transformed by the cochlea to electrical signals and delivered to the hearing nerves. In the brainstem and in the auditory cortex, neural generators produce the response EEG-signal to the sounds that are delivered to the ears. In U.S.-patent application Ser. No. 12/412343 these EEG-signals are recorded with a 2-channel EEG recorder from the following electrode positions: Left Mastoid—Vertex and Right Mastoid—Vertex. The EEG is averaged and a Fast Fourier Transform is applied to this average. A statistical test (F-test) compares the EEG response amplitude at 90 Hz with the EEG noise in the neighboring frequencies. In this way it is determined whether the subject or patient has good hearing abilities.

During hearing screening tests with the device as described in our U.S.-patent application with Ser. No. 12/412343, the headphone was placed easily on the head of the baby. However, the electrodes that were intended to make good electrical contact with the Mastoid positions were difficult to get on the right spot i.e. on the Mastoids of the baby. It was discovered by the inventor that this was mainly because the Mastoids of a baby have limited space, and secondly because the part of the scalp parietal to the Mastoids, has a curved shape instead of a flat surface needed to make secure electrical contact between electrodes and the scalp. In other words, the electrode-surface did not produce a good electrical contact to the scalp of the baby and therefore the EEG was not recorded well. The invention provides for a solution to these problems.

Furthermore, preliminary recordings in a neonatal clinic on neonates with a functional model of the hearing screener disclosed in U.S.-patent application Ser. No. 12/412343 revealed that mains power supplies at a distance of about 2.5 meters and less to our hearing screener, contributed significantly to 50 Hz noise. In fact these 50 Hz noise amplitudes were so large that they made hearing screening difficult, if not impossible. In our lab we were able to reproduce this electromagnetic noise interference by recording electrophysiological signals in the presence/absence of a mains cable that was plugged into the mains power supply. The distance of this mains cable to our hearing screener was less than 50 cm. Apparently, common mode suppression is not effective if the distance between the hearing screener and the EM-noise source is less than about 10 times the distance between the electrodes (=about 10 cm). Embodiments of the invention provide for a solution to these noise problems.

Although a large part of the description deals with hearing screening, the invention is applicable in a broader field, namely all fields where two EEG channels are measured with only three electrodes. Obviously more channels may be measured too (using further electrodes).

Potentially, all newborns worldwide (130 min/year) should be tested for a possible hearing loss by a hearing screener within the first four months after birth. Of course, only if there is a follow-up program, hearing screening makes sense. A hearing screener is intended to be used by e.g. a midwife or a nurse. Only a short training is needed to screen the ears of newborns correctly, no interpretation of test-results is needed: The outcome may be simply a pass or a refer. A pass means that hearing is OK. A refer means that additional hearing tests are needed. The nurse explains to the parents the screening procedure. After that, the nurse places the audio-transducers and the electrodes at the right positions of the child's head. The hearing is screened within about 2-10 minutes depending on state of sleep/rest/restlessness: sleep or rest reduces the test-time and improves a reliable outcome of the test significantly. After that, the audio-transducers and the electrodes are removed from the child's head and the nurse moves on to the next child. After a full day of hearing screening, the nurse may connect the screening device to a computer that is connected to a network such as the Internet to upload the screening outcomes of that day. Other nurses may also upload their screening results. In this way, all screening results may be transferred to one central computer. A software program may calculate the hearing screening statistics of that region/land or state (e.g. number of baby's tested, number of pass/refer).

In order to facilitate the discussion of the detailed embodiments a few expressions are defined hereinafter.

Throughout this description the term “galvanic isolation” should be interpreted such that there is no galvanic connection between the involved nodes which are galvanically isolated, i.e. that respective potentials are not related to each other.

For purposes of simplifying a description of the invention, the terms “operatively coupled”, “coupled” and formatives thereof as utilized herein refer to a connection between devices and/or portions thereof that enables operation in accordance with the invention.

Throughout this description the term “receiving the supply voltage” should be interpreted as receiving at least a first supply potential and a second supply potential different from the first potential. Alternatively, it may be interpreted as receiving a third supply potential different from the first supply potential and the second supply potential (for example a ground). The context of the description will make clear which situation is meant.

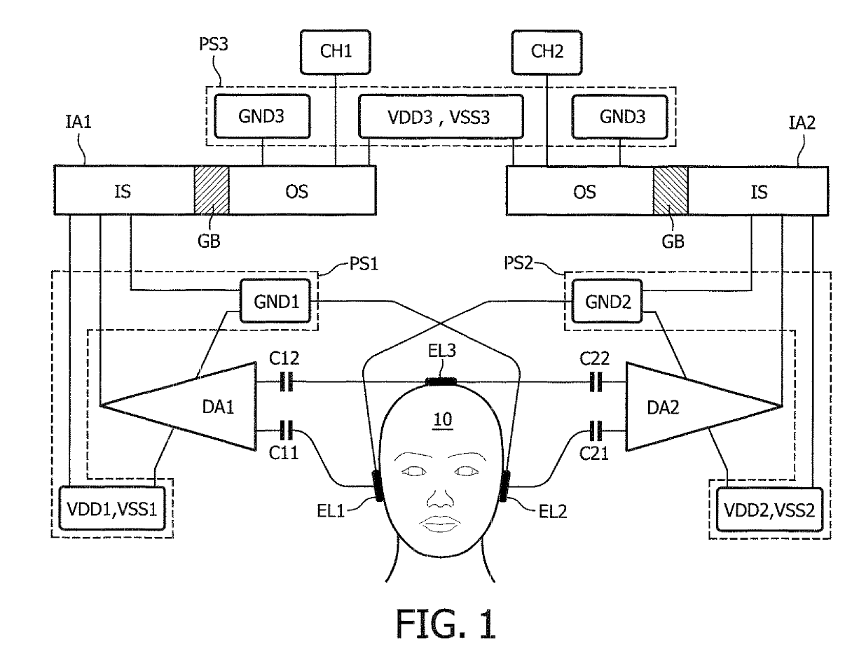

FIG. 1 shows a hearing screening system, one of the possible applications of the invention, in accordance with an embodiment of the invention. Notwithstanding the differences with the known systems, the part of the system as illustrated in this figure is also referred to as a brainstem recorder system or a hearing screener. FIG. 1 shows a head 10 of a patient or subject on which three electrodes is provided. A first electrode EL1 is provided at a location near the right ear (in a region between the right ear and the nose), a second electrode EL2 is provided at a location near the left ear (in a region between the left ear and the nose), and a third electrode EL3 is preferably provided at a location on top of the head (such as Cz or vertex). In accordance with the present invention the locations of the first electrode EL1 and the second electrode EL2 have been carefully selected. More information is given later in the description. The system in FIG. 1 constitutes a hearing screening system wherein a brainstem response is measured using two EEG channels CH1, CH2.

The first channel CH1 is measured as follows. A first differential amplifier DA1 is coupled to the first electrode EL1 and the third electrode EL3 and is arranged for amplifying the potential difference (first EEG-signal) between the first electrode EL1 and the third electrode EL3. In this embodiment the inputs of the respective amplifier DA1 are provided with coupling capacitors C11, C12. By doing so, the DC component of the voltage difference (which is a time varying quantity, i.e. a signal) is subtracted from the inputs. The inventor has realized that this allows the gain of the differential amplifier DA1 to be designed much larger, i.e. between 1000 and 200000 times, which is very beneficial for EEG signals, which are generally “drowned in noise”, i.e. the signals are very weak. It is not essential that decoupling capacitors are used on the input as a decoupling device, as long as a device or circuit is used which provides simultaneous DC-decoupling from and AC-coupling to the advantage of this embodiment is present.

The second channel CH2 is measured similarly. A second differential amplifier DA2 is coupled to the second electrode EL2 and to the third electrode EL3 and is arranged for amplifying the potential difference (second EEG-signal) between the second electrode EL2 and the third electrode EL3. In this embodiment the inputs of the respective amplifier DA2 are also provided with coupling capacitors C21, C22. By doing so, the DC component of the voltage difference is subtracted from the inputs.

The measurement of two EEG-channels in a brainstem recorder system or hearing screener system in accordance with the present system increases the reliability of the system. Nevertheless, the inventor has realized that better results can be achieved by using galvanically isolated power supplies for the respective differential amplifiers DA1, DA2. A first power supply PS1 is provided for supplying a first supply voltage VDD1, VSS1, GND1 to the first differential amplifier DA1. A second power supply PS2, which is galvanically isolated from the first power supply PS1, is provided for supplying a second supply voltage VDD2, VSS2, GND2 to the second differential amplifier DA2. The power supplies can be virtually any sort of power supply, but in an advantageous embodiment they comprise batteries. In this embodiment both power supplies are configured for respectively providing a first supply potential VDD1, VDD2, such as +15V, a second supply potential VSS1, VSS2, such as −15V, and an intermediate supply potential GND1, GND2, such as 0V (may be called ground, but this is an arbitrary choice). The supply voltages can be changed in accordance with the requirements of the circuit. In any case, the galvanic isolation between both channels reduces the noise generated by one channel which is induced in the other channel, and thereby increases the signal integrity of the system. This particular embodiment, however, goes further in improving the signal integrity.

A further improvement is obtained by coupling the intermediate supply potential GND1 of the first differential amplifier DA1 to the second electrode EL2, and by coupling the intermediate supply potential GND2 of the second differential amplifier DA2 to the first electrode EL1. In this way, the electrodes EL1, EL2 act as a corresponding input to the intermediate supply potential GND1, GND2. By doing so the intermediate supply potential GND1 of the first differential amplifier DA1 of the first channel CH1 moves along with the potential on the second electrode EL2, and the intermediate supply potential GND2 of the second differential amplifier DA2 of the second channel CH2 moves along with the potential on the first electrode EL1. This measure results in a clear common-mode rejection effect.

It must be noted that, instead, the respective ground levels could be connected to any other one of the electrodes. Nevertheless, such configuration suffers more from noise on the channels as the one illustrated in FIG. 1, i.e. the configuration in FIG. 1 is advantageous as experiments have shown that it provides very high signal integrity (noise reduction) on the channels. In these embodiments, the respective ground potentials are at least not connected to the same electrode as that would immediately couple the power supplies again. However, instead of coupling the ground potentials of the respective power supplies to one of the electrodes, also one of the other supply potentials VSS1, VSS2, VDD1, VDD2 could be taken (i.e. it is not essential to have a three potential power supply).

In the example of FIG. 1 both channels are effectively “brought together” (related to each other) by means of respective isolation amplifiers IA1, IA2. The isolation amplifiers each have a respective input side IS which each receive the respective supply potentials VSS1, VSS2, VDD1, VDD2, GND1, GND2 and respective output of the differential amplifiers. Further, the isolation amplifiers have a respective output side OS which is galvanically isolated from the respective input side IS by means of a galvanic barrier GB. Between the differential amplifiers DA1, DA2 and the isolation amplifiers IA1, IA2 filter circuitry may be added to improve the signal integrity.

Isolation amplifiers as such are well-known in the prior art. One of such known isolation amplifiers is the ISO122 from Burr-Brown Corporation. The respective output sides OS are both fed by a third power supply PS3, which is galvanically isolated from the first and second power supplies PS1, PS2. In this embodiment the third power supply is configured for providing a first supply potential VDD3, such as +15V, a second supply potential VSS3, such as −15V, and an intermediate supply potential GND3, such as 0V (may be called ground, but this an arbitrary choice). In accordance with an embodiment of the present system the two channels are brought together (coupled) at this point of the system. Alternatively, it may be done at another point in the flow. It is also possible that two individual processor units are coupled to the respective channels, and that the coupling is done thereafter.

It must be noted that, as mentioned in U.S. patent application Ser. No. 12/412343 the inventor was the first who provides a hearing screening system which only requires three electrodes to provide a dual-channel system. All prior solutions known so far need some kind of 4th reference electrode to one of the supply voltages of the differential amplifier (a conductive wrist band or at least one more electrode). Less electrodes in accordance with the present system means less cost, less handling time of the system and improved reliability of electrode contact to the skin of the subject (easier to use, faster to apply to a patient or subject, good quality EEG during the whole recording session).

Nevertheless, the present system is not restricted to three-electrode configurations only; it may be carried out with four electrodes or more. For example, the electrode on the Cz-position may be doubled (and kept spaced apart).

In this invention the method of measuring the EEG channels (part of hearing screen, but this is not essential) has been improved by an improved placement of the first and second electrodes EL1, EL2 on the head 10 of the subject or patient. The next figures are meant to illustrate this aspect of the invention in more detail.

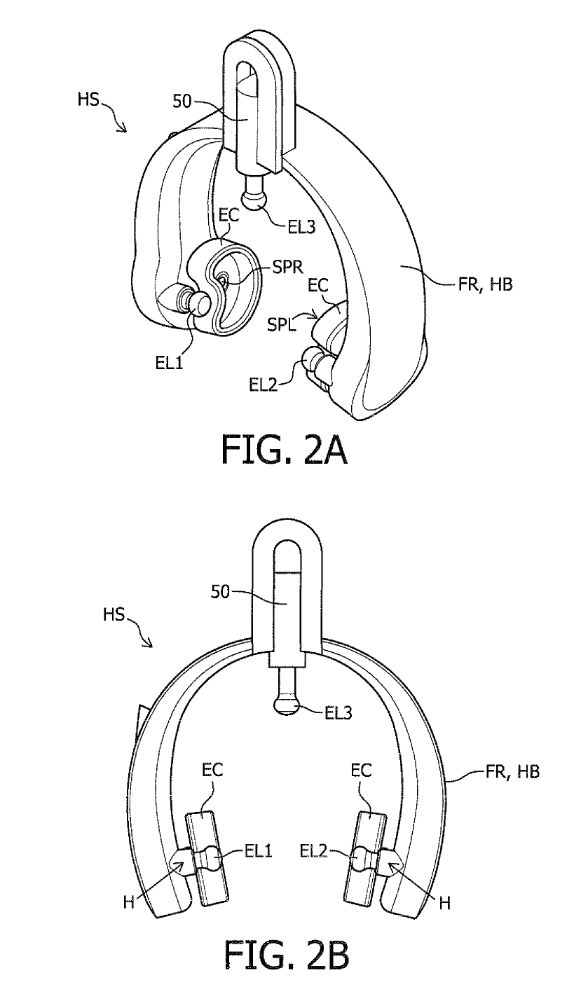

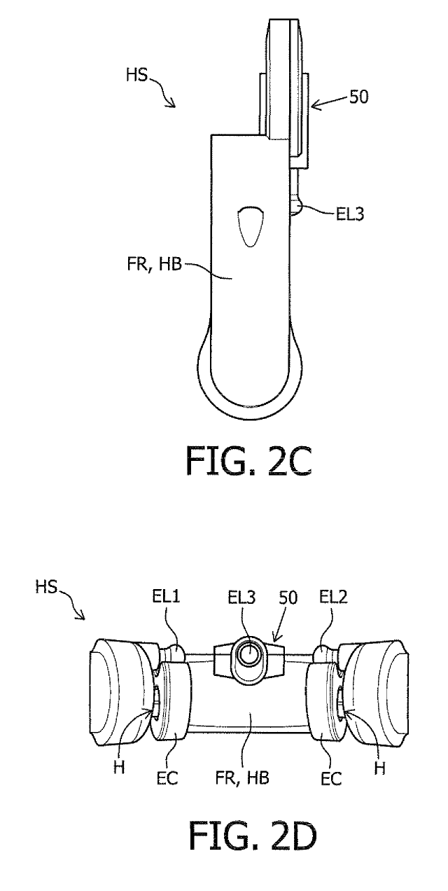

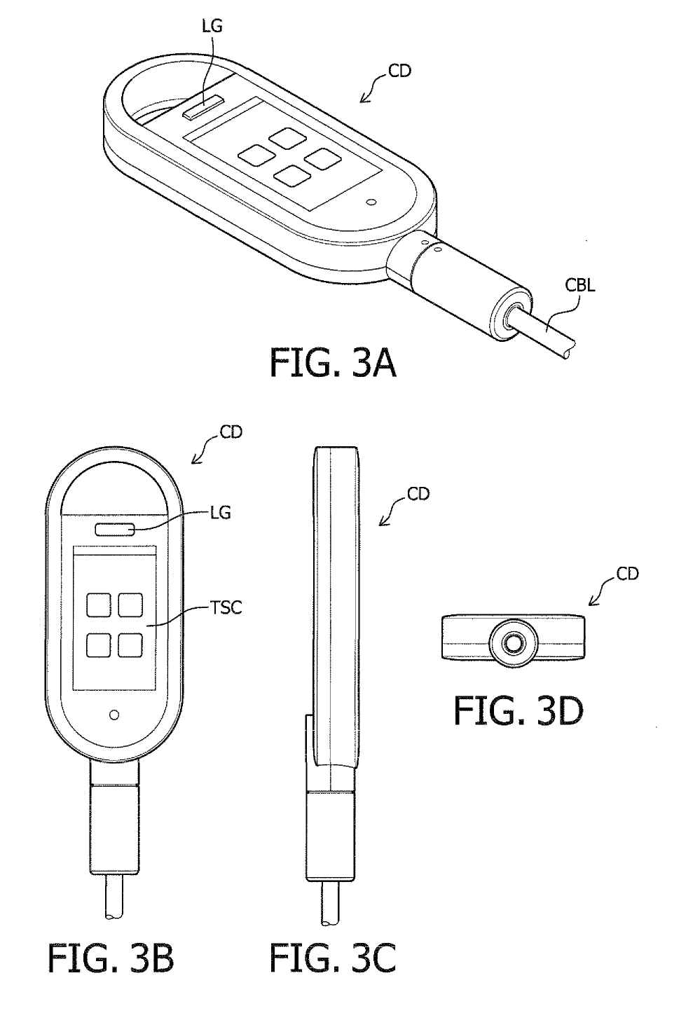

FIGS. 2a to 2d show different views of a head-set as part of a hearing screening system in accordance with another embodiment of the invention. FIG. 2a shows a 3D view of the head-set HS. FIG. 2b shows a front-view of the head-set HS. FIG. 2c shows a side-view and FIG. 2d shows a bottom-view. FIGS. 3a to 3d show different views of a remote-control to be coupled to the head-set of the hearing screening system of FIGS. 2a to 2d. The head-set HS comprises a frame FR (here a flexible head-band) with two ear caps EC, each ear cap EC provided at one end of the frame and being configured for receiving an ear of the head of the patient or subject (not shown in figure). The head-band FR is designed such that a wide range of head-sizes fit in the head-set. In this embodiment the ear-caps EC are connected to the head-band FR via hinges H which provide for some flexibility in the orientation of the ear-caps EC, but this is not essential. Also, the hinges H in accordance with an embodiment of the present system may be designed such that the ear-caps EC can be decoupled for cleaning/sterilizing purposes. Within each ear caps EC there is provided an auditory stimulus means SPL, SPR, such as a speaker or any other device which may be used to convert a signal into sound. The basic functions of the ear-caps EC are to guide sound emitted by the speakers to the ear channel in an optimal manner, and to prevent environmental sound to reach the ears. Ends of the head-band FR may also comprises microphones (not shown) integrated to record environmental sound. To that end, in accordance with an embodiment of the present system, these microphones may form a part of a noise-cancellation system (which may be integrated into the head-set too) which may be coupled to the speakers.

The head-set HS further comprises the first electrode EL1, the second electrode EL2, and the third electrode EL3. The first electrode EL1 and the second electrode EL2 are mounted on the frame FR such that, in operational use, they are positioned between the ears and the nose, whereas the third electrode is mounted such that, in operational use, it is positioned on a Cz-position of the head. In this embodiment the first electrode EL1 and the second electrode EL2 are positioned 1 cm to 2 cm from the ear (measured from the outer ear canal). For most subjects or patients this substantially coincides with the location of the joint between the jaw and the skull of the patient. However, many anatomic variations exist among humans and therefore also the ideal distance may deviate from person to person. In any case, there is quite a large area (more or less strip-shaped region) between the nose and each ear where the respective electrodes EL1, EL2 may be placed while still featuring a good signal-to-noise ratio of the measured EEG signal, and while still featuring easy integration with the head-set HS.

On the head-band FR there is further integrated an integrated amplifier comprising the block as illustrated and discussed with respect to FIG. 1. In order to make the head-set fit to larger number of patients or subjects the third electrode EL3 is illustratively shown mechanically coupled to the head-band HB via an adjustment device 50, which may be a spring structure in this example, but this is not essential. In accordance with this embodiment, the adjustment device provides a good connection of the third electrode EL3 and the Cz-position on the head of the patient or subject independent of the size of the head, and at the same time prevents the head-set to glide/slip from the head of the patient or subject (double function).

In an advantageous embodiment the electrodes may be durable electrodes. In the prior art a lot of electrodes are just thrown away after single use. The use of durable electrodes prevents such waste which is very advantageous for the environment. The electrodes may be formed from stainless steel, silver chloride, or sintered silver chloride, but other suitable materials are not excluded, for example Gold, Platinum, and Rubidium. The electrodes may be mounted to the head-set through an adjustable mounting device, such as springs, or other mountings that may be suitably applied. This embodiment facilitates that the electrodes automatically contact the skin of the subject or patient when the head-set is put on the head. The advantage of using springs is that apart from the mechanical flexibility, also the springs may provide a secure electrical path between the electrodes and the skin (e.g., the electrical current runs through the springs). Using a rigid headphone construction with durable electrodes instead of disposable ones, electrodes placed in front of the ears will produce a much better quality of EEG than if they were placed at the Mastoid positions. Using disposable electrodes connected to (non-rigid) leads will produce good quality EEG independent of the electrode position. However disposable electrodes have the disadvantage of high cost and consumption of natural resources, producing enormous amounts of waist.

With the head-set of the invention electrodes and sound transducers are attached to the subject or patient in just one move. This saves time in every single hearing screening attempt. All three electrodes are attached to the right position on the head, thus placement errors are strongly reduced.

For more details about the head-set HS reference is made to U.S. patent application Ser. No. 12/412343 which has been incorporated by reference in its entirety in this application.

In accordance with an embodiment of the present system, the hearing screening system (e.g., a brainstem recorder, a hearing screener headset, etc) may be coupled to a control device/remote control CD (see FIGS. 3a to 3d). In an embodiment this control device comprises processor unit for screening the hearing of a patient or subject and for rendering a result of testing (e.g., displaying a result, producing an auditory result, etc.). Apart from the processor unit the control device CD forms part of the user interface of the hearing screen system. The user interface comprises a touch screen TSC comprising operation buttons. The control device further comprises a region that is reserved for a logo LG. In this embodiment the control device CD is coupled to the headset via a cable CBL. However, the invention is not restricted to such wired solution. Alternatively, wireless solutions are also possible and do not depart from the scope of the invention as claimed.

The control device CD provides wireless data-transmission of hearing screening results to a central computer. This enables statistical evaluation of hearing screening results of a whole country or region. More information about the function of the control device is given later in this description.

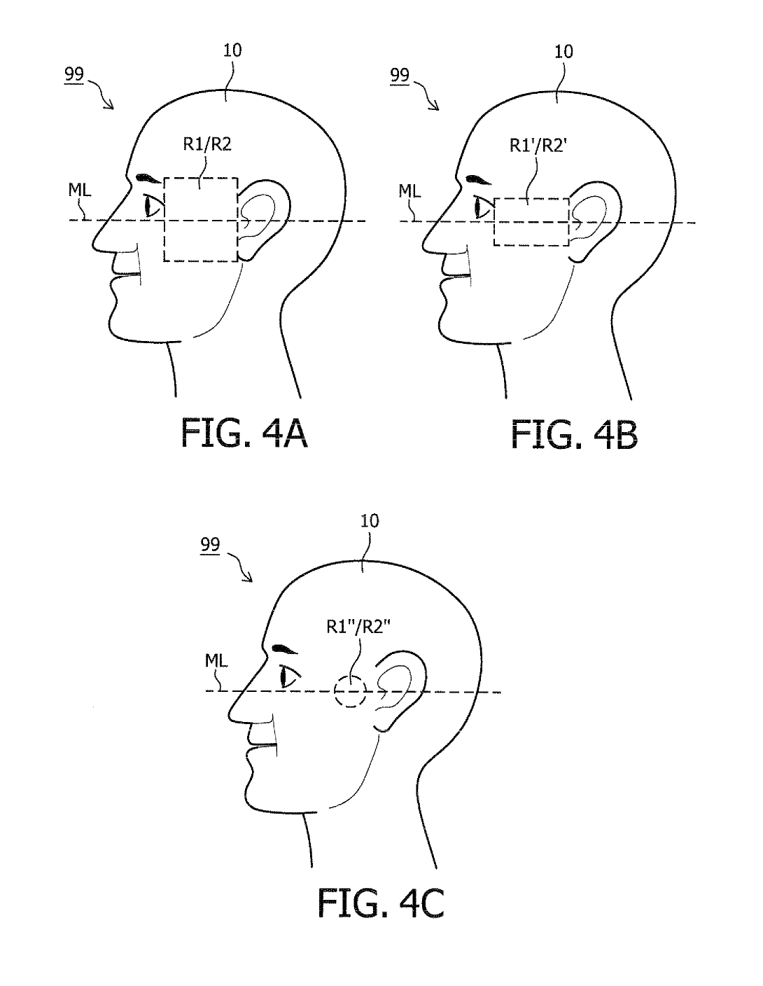

FIGS. 4a to 4c shows regions on a head of a subject of patient on which EEG measurement electrode are placed in accordance with different embodiments of the invention. As has already been mentioned the inventor has discovered that it is advantageous to place the first and second electrodes before the ears rather than behind the ears (on mastoids). Not only does this enable visual inspection of the electrode placement (when the subject or patient is lying on his/her back). Also this had lead to a more reliable measurement of the EEG signals and thereby a more reliable hearing screening method and system. In FIGS. 4a to 4c there is presented a subject or patient 99 having a head 10 with two ears (of which one is visible), two eyes (of which one is visible) and one nose. This complies with a healthy person having normal anatomic properties and dimensions. As most healthy persons have a substantially symmetrical head only one side of the head 10 has been illustrated. In general, in accordance with the invention the first and second electrodes are placed between each respective ear and respective eye on each side of the head. The next figures disclose advantageous embodiments of the method (and corresponding systems and devices).

FIG. 4a schematically illustrates a location of the first and second electrodes in accordance with a first embodiment of the invention. In this figure the first region R1 and second region R2 for the first electrode are strip-shaped and substantially between the respective ear and the respective eye. The strip has a (virtual) midline ML which extends from an external auditory meatus of the respective ear to the nose (as illustrated in the figure). The strip has a width of about 6 centimeters.

FIG. 4b schematically illustrates a location of the first and second electrodes in accordance with a second embodiment of the invention. This figure illustrates the first (further restricted with respect to FIG. 4a) region R1 and the second (further restricted with respect to FIG. 4a) region R2, wherein the strip has a width of about 4 centimeters. In a further embodiment the strip has a width of about 2 centimeters. Positioning the respective electrodes within this region further facilitates easy integration in the headset, but at the same time enables a better contact between the electrodes and the skin and also better visual inspection during the measurements. The restricted regions as illustrated in FIG. 4b generally more coincide with the cheek bone and are very suitable for placing electrodes.

FIG. 4c schematically illustrates a location of the first and second electrodes in accordance with a third embodiment of the invention. This figure illustrates the first (further restricted with respect to FIG. 4b) region R1 and the second (further restricted with respect to FIG. 4b) region R2, wherein the strip has a width of about 2 centimeters. The first (even further restricted) region R1″ and the second (even further restricted) region R2″ substantially coincide with a location of the joint between the skull and the jaw. Experiments have shown that this region is particularly advantageous for positioning an electrode, while obtaining a very good contact (and thereby a very good signal-to-noise ratio).

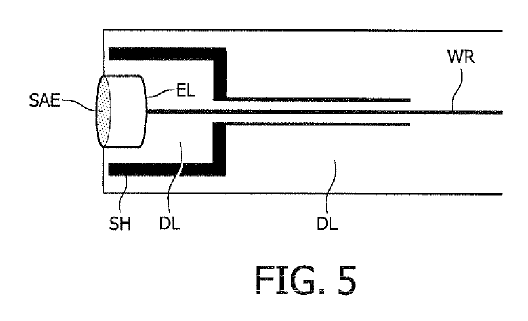

FIG. 5 shows a shielded electrode in accordance with yet another embodiment of the invention. The shielded electrode comprises an electrode EL having a sensing area SAE which is connected to a signal wire WR (which is on its turn coupled to an amplifier). An electrically conductive shield (electromagnetic shield) SH is provided covering the electrode EL and extending almost to the sensing area SAE, but being electrically insulated there from by a dielectric DL. Thus only the sensing area of the electrode is not EM-shielded, because this surface is for contacting the skin in operational use. Furthermore, the electrically conductive shield SH extends to and is electrically coupled to the shielding of the wire WR. Experiments have shown that this configuration effectively shields the electrode and signal wire WR from EM radiation, which leads to a much better signal-to-noise ration. The electromagnetic shield is electrically isolated from the signal source (e.g. the skin of a human or other (bio)-potential source). The electromagnetic shield SN is connected to a reference potential, which can be, but is not necessarily, galvanically connected to the power supply of the amplifier. In the amplifier design presented in FIG. 1 the electromagnetic shield SH is preferably connected to the third ground potential GND3.

It must be noted that the application of shielded electrodes as such is independent from the electrode configuration, i.e. the number of electrodes and the electrode positions). Thus this aspect of the invention may also be claimed in a broader technical field. It must be further noted that a Faraday cage and its electro-magnetic (EM) shielding properties as such, is well known from prior art. Hence, further details of on the implementation of the shields are not given in this description.

The Automated Auditory Brainstem Response (AABR) is a special form of Auditory Brainstem Response (ABR). In U.S.-patent application with Ser. No. 12/412343 a device is described that is placed on the head of the subject that records a two-channel EEG from the following electrode positions: Left Mastoid—Cz (=Vertex) and Right Mastoid—Cz (=Vertex). Other electrode positions that are commonly used to record the ABR or other auditory evoked potentials include: Neck, Inion, Nasion, Fpz, Fz, Cz (=Vertex), Pz, Oz, P3, P4, F3, F4, T3,T4, C3,C4, A1,A2 (see New Handbook of Auditory Evoked Responses, FIG. 3.9) and “Improving the signal to noise ratio of the Auditory Steady-State Response” by C. S. van der Reijden page 41, 58.

The hearing screening system (headset/brainstem recorder) in accordance with an embodiment of the present system may be completed as follows. The head-set is placed on the head 10 of a subject or patient, for example a newborn. The two-channel electrophysiological signals are measured and amplified for example, 15,000 times (as discussed earlier) by the two-channel EEG amplifier that is integrated into the head-set. The amplified signals are transmitted to the processor unit. In this example embodiment, the processor unit may include a powerful and energy-saving computer, such as an XSCALE PXA310 (624 MHz clock-frequency) from Toradex. More information on the PXA310 from Toradex is to be found in the datasheets, which are available at www.toradex.com.

In accordance with an embodiment of the present system a computer program stored in a memory configures the computer (e.g., a processor) to generate a data-array, for example, with 92 clicks per second for the right ear and 90 clicks per second for the left ear. A click in electrical form may be in a form of a square pulse of 100 microseconds in width. The UCB1400-chip on the PXA310-module has a headphone-buffer. Because of this, the computer produces enough power to drive a headphone without additional (external) buffering. Therefore, the stereo-audio line output of the PXA310 may be directly connected to the speakers SPL, SPR in the head-set. The speakers SPL, SPR of the head-set transform the electrical clicks into acoustical stimuli, for example of about 1 msec in length and with a loudness 35 dB nHL (normalized hearing level) being the international accepted loudness level of audible stimuli for hearing screening. The amplified two-channel EEG may be connected to the stereo-audio line input. The stereo-line in signals (EEG) may be analog-digital converted, for example, in synchrony with digital-analog conversion of the stereo-line out signals, such as in exact synchrony. In accordance with this embodiment, the synchronous conversion is a feature for successful detection of evoked responses. This feature has been tested thoroughly. In accordance with an embodiment of the present system, the methods provided by a suitably programmed processor by software to detect responses to the auditory stimuli may be similar to the methods described in the article of John M S and Picton T W, “MASTER: a Windows program for recording multiple auditory steady-state responses”, Computer methods in Biomedicine 2000; 61, 125-150. This document is hereby incorporated by reference in its entirety.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory coupled to the processor. As readily appreciated, application data (computer programming software) and other data are received by the processor for configuring the processor to perform operation acts in accordance with the present system. The operation acts include controlling at least one of the auditory stimulus devices to generate an auditory stimulus and to receive responses from one or more electrodes in accordance with the present system. Further, the processor may be suitably programmed to correlate responses locally and/or transmit responses to a remote system for correlation and to cause rendering (e.g., display) of a result (e.g., pass/refer).

An illustrative example of operation of the present system may be provided as follows. In exact synchrony with the audible clicks, the two-channel EEG is accepted. Small periods of 512 ms EEG, so-called epochs, are tested for excessive EEG noise or ambient (audible) noise. If excessive noise is present, this epoch is rejected. If the EEG noise level is below a certain limit, such as about 20-30 pV, and if the ambient noise level inside of the ear-caps is below a certain limit, such as about 25 dB (but this may also be higher), then the epoch is accepted. Sixteen consecutive accepted epochs (16×512=8192 ms of EEG) are put into an array. A Fast Fourier Transform is applied to this array. If responses to the auditory stimuli are present, they will appear as a sharp peak at exactly the repetition rate (90 Hz or 92 Hz) of the audible stimulus (John et al., 2000, pp 127). An F-test (F-ratio) estimates the probability that a response at a certain frequency (90 Hz or 92 Hz) is significantly above the neighboring frequencies (noise level) (John et al., 2000, pp 127). The significance level can be set at any pre-defined value (e.g. 1%, 0.1% or 0.05%). The result of the F-test (significant/not significant above noise level) is displayed on the computer-unit as a pass/refer. After the first sixteen epochs have been accepted, recordings continue. The next series of epochs is averaged with the first series followed by the computational methods as described above. And so on, till a pass is displayed for the left and the right ear or until a predefined number of accepted epochs (for example, about 512) has been reached.

The computer may provide a user interface, such as through use of a touch screen and/or another display device. Relevant data of the newborns (or other patients or subjects) to be tested may be displayed and controlled through this interface and through suitably programming of a processor of the computer. Progress during hearing-screening (recording time, number of accepted epochs), pass/refer and noise levels may be displayed on the screen.

After a full day of hearing screenings, the results that have been collected in one or more hearing screeners, can be transferred to a central computer. Software running on this central computer may program the processor to calculate statistics of pass/refer rates of a region, state or land. This enables a day by day tracking of the hearing screening results. This software has already been developed in our lab and is in use in Belgium at “Kind en Gezin” and at Depistage Surdité.

Various variations of the communication system in accordance with the invention are possible and do not depart from the scope of the invention as claimed.

The invention provides a method of measuring an electroencephalogram signal in response to an auditory stimulus applied to a subject or patient 99, wherein the method uses at the most a first electrode EL1, a second electrode EL2, a third electrode EL3. The method comprises: i) positioning the first electrode EL1 on the head 10 of the patient or subject 99 in a first region R1 extending substantially between a right ear and a right eye; ii) positioning the second electrode EL2 on the head 10 of the subject or patient 99 in a second region R2 extending substantially between a left ear and a left eye; iii) positioning the third electrode EL3 on the head 10 of the subject or patient 99 in a third region Cz, the third region Cz being different from the first region R1 and the second region R2; iv) applying the auditory stimulus to at least one ear of the subject or patient 99; v) measuring a first potential difference between the first electrode EL1 and the third electrode EL3 in response to the auditory stimulus to obtain a first electroencephalographic signal, and measuring a second potential difference between the second electrode EL2 and the third electrode EL3 in response to the auditory stimulus to obtain a second electroencephalographic signal. The invention further provides a method of hearing screening a subject or patient, comprising such method. The invention also provides a measurement system (such as a hearing screener) for carrying out such method and an apparatus (head-set) for use in such system. The invention provides for an improved method of measuring an electroencephalogram signal in response to an auditory stimulus applied to a subject or patient, in particular where two channels are measured using only three electrodes.

The invention may be applied in various application areas. For example, the invention may be applied in any method or system for measuring a bio-potential on the skin, such as hearing screening methods and systems.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage. Throughout the Figures, similar or corresponding features are indicated by same reference numerals or labels.

1. A method of measuring an electroencephalogram signal in response to an auditory stimulus applied to a subject or patient (99), wherein the method uses at the most a first electrode (EU), a second electrode (EL2), a third electrode (EL3), the method comprising:
positioning the first electrode (EU) on the head (10) of the patient or subject (99) in a first region (R1) extending substantially between a right ear and a right eye;
positioning the second electrode (EL2) on the head (10) of the subject or patient (99) in a second region (R2) extending substantially between a left ear and a left eye;
positioning the third electrode (EL3) on the head (10) of the subject or patient (99) in a third region (Cz), the third region (Cz) being different from the first region (R1) and the second region (R2);
applying the auditory stimulus to at least one ear of the subject or patient (99);
measuring a first potential difference between the first electrode (EU) and the third electrode (EL3) in response to the auditory stimulus to obtain a first electroencephalographic signal, and
measuring a second potential difference between the second electrode (EL2) and the third electrode (EL3) in response to the auditory stimulus to obtain a second electroencephalographic signal.

2. The method as claimed in any claim 1, wherein at least one of the first region (R1) and the second region (R2) is further defined as a fictitious strip having a midline (ML) extending from an external auditory meatus of the respective ear in a direction towards a nose.

3. The method as claimed in claim 2, wherein the strip has a width of 6 centimeters, and preferably 4 centimeters, and even more preferably 2 centimeters.

4. The method as claimed in claim 3, wherein a heart-heart distance between each respective one of the first and second electrodes (EL1, EL2) and the respective external auditory meatus lies in the range between 1 and 2 centimeters.

5. The method as claimed in claim 1, wherein the third electrode (EL3) is positioned on the midline of the head of the subject or patient.

6. The method as claimed in claim 5, wherein the third electrode (EL3) is positioned on a Cz position of the head (10).

7. A method of hearing screening a subject or patient (99), the method comprising the method as claimed in claim 1.

8. A measurement system for carrying out the method as claimed in claim 1.

9. The measurement system as claimed in claim 8, further comprising a frame (FR), and the respective electrodes (EL1, EL2, EL3), the respective electrodes (EL1, EL2, EL3) being mounted to the frame (FR), wherein the frame (FR) is further configured for fixing respective positions of the respective electrodes in the respective regions (R1, R2, R1′, R2′, R1″, R2″, Cz) in operational use.

10. The measurement system as claimed in claim 9, further comprising an auditory stimulus device (SPL, SPR) for applying the auditory stimulus to the at least one ear of the subject or patient, the auditory stimulus device (SPL, SPR) being mounted to the frame (FR).

11. A hearing screener comprising the measurement system as claimed in claim 10, and further comprising an amplifier for amplifying the electroencephalogram signals, and a processor unit connected to the auditory stimulus device (SPL, SPR) for controlling the application of the auditory stimulus, the processor unit being further configured for receiving, collecting, and processing data from the amplifier to obtain quantitative data about the hearing ability of the subject or patient.

12. An apparatus (HS) for use in the measurement system of claim 9, the apparatus comprising the frame (FR), the first electrode (EU), the second electrode (EL2), and the third electrode (EL3).

13. The apparatus (HS) as claimed in claim 12, further comprising an auditory stimulus device (SPL, SPR) for applying the auditory stimulus to the at least one ear of the subject or patient (99).

14. The apparatus (HS) as claimed in claim 13, wherein the auditory stimulus device (SPL, SPR) has been mounted to the frame (FR).

15. The apparatus (HS) as claimed in claim 12, wherein at least one of the respective electrodes (ELL EL2, EL3) comprises an electrically conductive shield (SH) being electrically insulated from the at least one of the respective electrodes and covering a substantial part of the at least one of the respective electrodes and extending substantially to a contact surface of the at least one of the respective electrodes, wherein the electrically conductive shield (SH) is connected to a reference potential (VDD1, VSS1, VDD2, VSS2, VDD3, VSS3) of the measurement system.
